(19) United States
(12) Patent Application Publication
(10) Pub. No.: US 2022/0248866 A1
(43) Pub. Date: Aug. 11, 2022

(54) CONTROL METHOD, RECORDING MEDIUM, AND CONTROL APPARATUS

(71) Applicant: Panasonic Intellectual Property Management Co., Ltd., Osaka (JP)

(72) Inventors: Yasuo KOHASHI, Osaka (JP); Etsuko MIZUNO, Osaka (JP); Hiroko SUGIMOTO, Kyoto (JP)

(21) Appl. No.: 17/630,315

(22) PCT Filed: Feb. 15, 2021
(86) PCT No.: PCT/JP2021/005551

§ 371 Date: Jan. 26, 2022

(30) Foreign Application Priority Data
Feb. 27, 2020 (JP) 2020-032063

(51) Int. Cl. A47C 21/04; A61B 5/00

(52) U.S. Cl. CPC A47C 21/04; A61B 5/6892; A61B 5/4809

(57) ABSTRACT

The control method according to one aspect of the present disclosure is a method of controlling an apparatus which adjusts a bed ambient temperature for a user, the method including obtaining one of measurement information in a sensor and setting information of the apparatus; estimating the bed ambient temperature and one of getting in bed, starting of sleep, and falling asleep by the user, based on the one of the measurement information and the setting information; and when a first temperature is the bed ambient temperature before the user gets into bed, starts sleeping, or falls asleep, changing the bed ambient temperature to a second temperature, which is lower than the first temperature by 0.5° C. or more, in a period after the user gets into bed, starts sleeping, or falls asleep and before a non-shallow sleep state of the user ends.

### CROSS-REFERENCE OF RELATED APPLICATIONS

This application is the U.S. National Phase under 35 U.S.C. § 371 of International Patent Application No. PCT/JP2021/005551, filed on Feb. 15, 2021, which in turn claims the benefit of Japanese Application No. 2020-032063, filed on Feb. 27, 2020, the entire disclosures of which Applications are incorporated by reference herein.

### TECHNICAL FIELD

The present disclosure relates to a control method for an apparatus which adjusts a bed ambient temperature for a user, a recording medium, and a control apparatus for the apparatus.

### BACKGROUND ART

PTLs 1 to 4 disclose a method of directly controlling the body temperature of a user when the user falls asleep or is sleeping and a method of bringing the user into a comfortable sleep state by controlling the temperature of an environment where the user is present.

### CITATION LIST

### Patent Literature

[PTL 1] Japanese Patent No. 4228974
[PTL 2] Japanese Unexamined Patent Application Publication No. 2009-264704
[PTL 3] Japanese Patent No. 2987981
[PTL 4] Japanese Unexamined Patent Application Publication No. 2005-296642

### SUMMARY OF INVENTION

### Technical Problem

The present disclosure provides a control method and the like which can bring the user into a more comfortable sleep state than in the related art.

### Solution to Problem

The control method according to one aspect of the present disclosure is a control method for an apparatus which adjusts a bed ambient temperature for a user, the control method including: obtaining one of measurement information in a sensor and setting information of the apparatus; estimating the bed ambient temperature and one of getting in bed, starting of sleep, and falling asleep by the user, based on the one of the measurement information and the setting information; and when a first temperature is the bed ambient temperature before the user gets into bed, starts sleeping, or falls asleep, changing the bed ambient temperature to a second temperature, which is lower than the first temperature by 0.5° C. or more, in a period after the user gets into bed, starts sleeping, or falls asleep and before a non-shallow sleep state of the user ends.

The recording medium according to one aspect of the present disclosure is a non-transitory computer-readable recording medium for use in a computer, the recording medium having a computer program recorded thereon for causing the computer to execute the control method.

The control apparatus according to one aspect of the present disclosure is a control apparatus for an apparatus which adjusts a bed ambient temperature for a user, the control apparatus including: an obtainer which obtains one of measurement information in a sensor and setting information of the apparatus; an estimator which estimates the bed ambient temperature and one of getting in bed, starting of sleep, and falling asleep by the user, based on the one of the measurement information and the setting information; and a controller which causes the apparatus to change the bed ambient temperature to a second temperature, which is lower than a first temperature by 0.5° C. or more, in a period after the user gets into bed, starts sleeping, or falls asleep and before a non-shallow sleep state of the user ends, when the first temperature is the bed ambient temperature before the user gets into bed, starts sleeping, or falls asleep.

These comprehensive or specific aspects may be implemented with a system, a method, an integrated circuit, a computer program, or a computer-readable recording medium such as a CD-ROM, or may be implemented with any combination of a system, a method, an integrated circuit, a computer program, and a recording medium.

### Advantageous Effects of Invention

The control method according to one aspect of the present disclosure and the like can bring the user into a more comfortable sleep state than in the related art.

### BRIEF DESCRIPTION OF DRAWINGS

FIG. 1 is a diagram illustrating the outline of a control system included in the control apparatus according to an embodiment.

FIG. 2 is a block diagram illustrating a configuration of the control apparatus according to the embodiment.

FIG. 3 is a graph schematically illustrating one example of the sleep cycle.

FIG. 4 is a flowchart illustrating a processing procedure in the control apparatus according to the embodiment.

FIG. 5 is a flowchart illustrating details of the temperature control processing procedure in the control apparatus according to the embodiment.

FIG. 6 is a graph illustrating a first example of the time change in bed ambient temperature controlled by the control apparatus according to the embodiment.

FIG. 7 is a graph illustrating a second example of the time change in bed ambient temperature controlled by the control apparatus according to the embodiment.

FIG. 8 is a graph illustrating a third example of the time change in bed ambient temperature controlled by the control apparatus according to the embodiment.

FIG. 9 is a graph illustrating the experimental result of the time change in sleep depth of a user and the time change in bed ambient temperature controlled by the control apparatus according to the embodiment.

FIG. 10 is a graph illustrating another example of the experimental result of the time change in sleep depth of the user in the control apparatus according to the embodiment.

FIG. 11 is a graph illustrating another example of the experimental result of the time change in sleep depth of the user according to Comparative Example.

### DESCRIPTION OF EMBODIMENTS

### (Underlying Knowledge Forming Basis of the Present Disclosure)

In the related art, there are techniques of controlling the temperature for bringing a user into a comfortable sleep state or for promoting a good sleep of the user. Examples thereof include a technique of keeping a room in which the user sleeps at a constant room temperature. Examples thereof also include a technique of changing the room temperature in a V-shape such that the room temperature is decreased to an intermediate time of the total sleep hours of the user and then is increased from the intermediate time. Other examples thereof include a technique of decreasing the room temperature for 180 minutes from the time when a user in bed falls asleep, maintaining the room temperature for 60 minutes, and then increasing the room temperature, for example.

The room temperature control in the related art can induce drowsiness of the user at night when the user falls asleep (or sleeps) by decreasing the temperature of the room where the user sleeps. On the other hand, in the morning when the user awakes (or wakes up), the user can be activated (or can be readily awaken) by increasing the temperature of the room where the user sleeps.

Thus, control of the room temperature of the room where the user sleeps can bring the user into a comfortable sleep state.

Here, the 90-minute cycle of non-shallow sleep and shallow sleep generated by the biological clock (also referred to sleep cycle or circadian rhythm) is important to bring the user into a comfortable sleep state. In particular, in users who cannot have a comfortable sleep state, the sleep cycle may not be generated well in some cases. The user can have a comfortable sleep by alternation of the non-shallow sleep and the shallow sleep which appropriately occurs. Accordingly, the user can be brought into a comfortable sleep state by controlling appropriate occurrence of alternation of the non-shallow sleep and the shallow sleep.

The present inventors, who have conducted extensive research, have found a method which can cause the user to appropriately fall asleep and can bring the user into a more comfortable sleep state by causing the sleep cycle to appropriately occur in the user.

An embodiment will now be described in detail with reference to the drawings as needed. To be noted, detailed descriptions beyond necessity will be omitted in some cases. For example, detailed descriptions of things already well known and overlapping descriptions of substantially identical configurations will be omitted in some cases. This is for avoiding unnecessary redundancy of the description below to facilitate understanding by persons skilled in the art.

The present inventors provide the accompanying drawings and the description below for sufficient understanding of the present disclosure by persons skilled in the art, and do not intend to limit the subjects to be described to Claims according to these.

In the description below, the state where the user is in bed and is doing a task such as reading with the eyes open may be referred to “in bed” (a conscious state) in some cases. The state where the user is in bed, closes the eyes without doing any task such as reading, and decides to sleep may be referred to starting of sleep (a conscious state) in some cases. The state where the user falls asleep may be referred to as asleep (a non-conscious state) in some cases. The state where the user wakes up may be referred to as awake in some cases.

In the description below, specific numeric values of time or temperature will be described. For example, “60 minutes” not only means exactly 60 minutes, but also includes errors of about several percent. The same is also applied to other numeric expressions.

### Embodiment

### [Configuration]

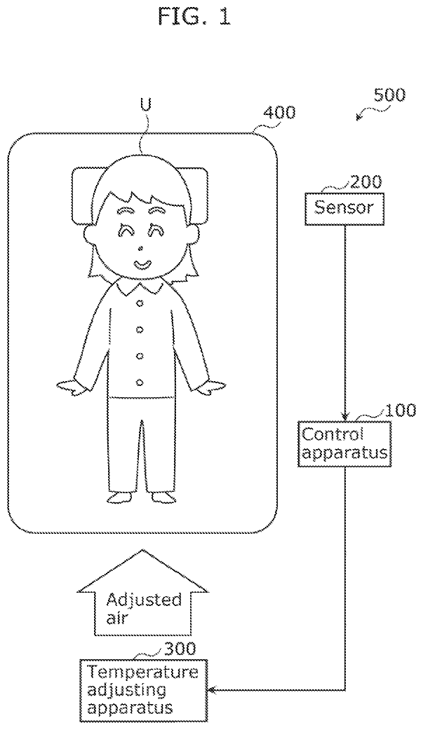

FIG. 1 is a drawing for illustrating the outline of control system 500 including control apparatus 100 according to an embodiment.

Control system 500 is a system which controls comfortable sleep of user U by adjusting (controlling) the temperature around bed 400, which is equipment or furniture for sleeping used by user U during sleep, such as a futon or a bed (hereinafter, the temperature around bed 400 is also simply referred to bed ambient temperature).

For example, control system 500 includes control apparatus 100, sensor 200, and temperature adjusting apparatus 300. In control system 500, control apparatus 100 controls the temperature of the environment surrounding bed 400 (e.g., room temperature) of user U who starts sleeping, by controlling temperature adjusting apparatus 300, based in the ambient temperature of the bed of user U measured by sensor 200, for example. Thereby, control apparatus 100 controls such that user U has comfortable sleep states when user U gets into bed, is sleeping, and wakes up (awakes).

Control apparatus 100 may control the temperature of the environment surrounding bed 400 (e.g., room temperature) of user U who gets into bed or falls asleep, rather than who starts sleeping. Hereinafter, control apparatus 100 will be described assuming that control apparatus 100 controls the temperature of the environment surrounding bed 400 of user U who starts sleeping.

Sensor 200 includes a thermometer for measuring the ambient temperature of the bed of user U, for example. Sensor 200 is communicably connected with control apparatus 100, and outputs temperature information indicating the measured temperature to control apparatus 100.

Sensor 200 (more specifically, a thermometer included in sensor 200) is arranged in the bed surroundings. The bed surroundings indicate surroundings of bed 400 which user U uses for sleeping, and indicate an area near the head of the bed of user U, for example. For example, the temperature in a futon having a large thickness hardly changes even when the room temperature is changed. For this reason, for example, the temperature of the body of user U covered with the futon hardly changes even when the room temperature is changed. Thus, control apparatus 100 causes temperature adjusting apparatus 300 to adjust the temperature around the face of user U, for example. Thereby, control apparatus 100 promotes comfortable sleep of user U by changing the temperature around the face of user U. For this reason, for example, the thermometer included in sensor 200 is arranged near the head of the bed close to the face of user U. For example, the thermometer included in sensor 200 may be arranged vertically above bed 400 (e.g., at a position vertically above about 50 cm from bed 400). In such a configuration, using sensor 200, control apparatus 100 can appropriately measure the temperature of the position which effectively affects user U.

Sensor 200 may include not only the sensor for measuring the temperature, such as the thermometer, but also a plurality of other sensors. For example, sensor 200 may include a sensor which measures the starting of sleep by user U in bed 400. The sensor can be any sensor, and may be a pressure sensor arranged in the bed, for example. Sensor 200 outputs sleep-start information to control apparatus 100, for example, the sleep-start information indicating that user U starts sleeping.

Thus, sensor 200 transmits pieces of measurement information obtained from measurement, such as the temperature information and the sleep-start information, to control apparatus 100.

Temperature adjusting apparatus 300 is an apparatus which adjusts the ambient temperature of the bed of user U. Temperature adjusting apparatus 300 is controlled by control apparatus 100 to release air at a predetermined timing, the air being adjusted to have a predetermined temperature. In the present embodiment, temperature adjusting apparatus 300 is an air-conditioning apparatus (air conditioner).

Temperature adjusting apparatus 300 suffices to enable adjustment of the ambient temperature of the bed of user U, and is not limited to the air-conditioning apparatus. Temperature adjusting apparatus 300 may be an electric blanket or a Peltier device.

Control apparatus 100 controls temperature adjusting apparatus 300 which adjusts the ambient temperature of the bed of user U. Control apparatus 100 is a computer, for example. Control apparatus 100 is communicably connected to sensor 200 and temperature adjusting apparatus 300. Control apparatus 100 may be communicably connected to sensor 200 and temperature adjusting apparatus 300 wiredly or wirelessly.

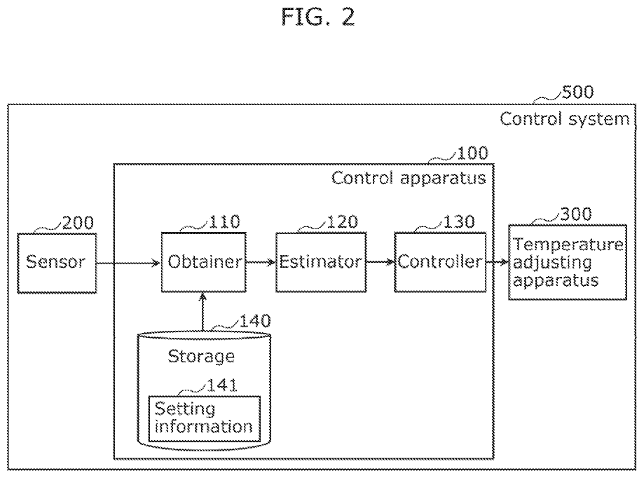

FIG. 2 is a block diagram illustrating the configuration of control apparatus 100 according to the embodiment.

Control apparatus 100 includes obtainer 110, estimator 120, and controller 130.

Obtainer 110 is a communication interface which obtains the measurement information from sensor 200. For example, obtainer 110 is a communication adaptor connected to a communication cable (not illustrated) connected to sensor 200.

For example, controlling of the bed ambient temperature by temperature adjusting apparatus 300 and the bed ambient temperature have a predetermined relation in some cases. In such cases, for example, by controlling temperature adjusting apparatus 300, control apparatus 100 can control the bed ambient temperature to an appropriate temperature without the temperature information from sensor 200, which indicates the bed ambient temperature. In such a case where control apparatus 100 is preliminarily provided with temperature relation information indicating the relation between control of the bed ambient temperature by temperature adjusting apparatus 300 and the bed ambient temperature, control apparatus 100 may control temperature adjusting apparatus 300 based on the temperature relation information.

In some cases, for example, user U may operate temperature adjusting apparatus 300 to set the timing of stopping the operation of temperature adjusting apparatus 300 at a timing when user U starts sleeping. In other words, user U predetermines the time when user U starts sleeping. In this case, for example, even if control apparatus 100 does not obtain the sleep-start information from sensor 200, the sleep-start information indicating that user U starts sleeping, control apparatus 100 can estimate the starting of sleep by user U based on the sleep start time information indicating the predetermined time when user U starts sleeping, and can start control of temperature adjusting apparatus 300.

Thus, control apparatus 100 obtains one of the measurement information in sensor 200 and setting information 141 of temperature adjusting apparatus 300, and controls temperature adjusting apparatus 300 based on the obtained information. Obtainer 110 obtains the one of the measurement information in sensor 200 and setting information 141 of temperature adjusting apparatus 300.

Setting information 141 may also be obtained from an external apparatus communicably connected to control apparatus 100, temperature adjusting apparatus 300, or the like.

Control apparatus 100 may also include an operation unit for obtaining setting information 141 from user U, such as buttons, a keyboard, or a touch panel operated by user U. As setting information 141, control apparatus 100 may store information accepted by the operation unit in storage 140.

In the case where control apparatus 100 controls temperature adjusting apparatus 300 based on setting information 141 including the temperature relation information and the sleep start time information, rather than the measurement information obtained from sensor 200, control system 500 may not include sensor 200.

Obtainer 110 obtains third temperature information including a third temperature which is a temperature when user U is sleeping, for example. The third temperature information is information including the third temperature which is a temperature (optimal temperature) at which user U has a comfortable feeling during sleeping, for example.

Obtainer 110 obtains wake-up setting information including wake-up setting time of user U (i.e., the time at which user U wakes up), for example.

Obtainer 110 obtains the third temperature information and the wake-up setting information from the operation unit, which accepts the operation from user U, for example.

Estimator 120 is a processor which estimates (calculates) the bed ambient temperature and one of getting in bed, starting of sleep, and falling asleep by user U (for example, the timing at which user U gets into bed, such as in-bed time, and in the present embodiment, the timing at which user U starts sleeping), based on one of the measurement information and setting information 141 obtained by obtainer 110.

For example, estimator 120 is implemented with a control program stored in storage 140 and the central processing unit (CPU) which executes the control program.

Controller 130 is a processor which changes the bed ambient temperature to a second temperature which is lower than the first temperature by 0.5° C. or more by controlling temperature adjusting apparatus 300, in a period after user U gets into bed, starts sleeping, or falls asleep (in the present embodiment, after user U starts sleeping, more specifically, at a timing when user U closes eyes and starts sleeping) and before the non-shallow sleep state of user U ends (that is, before user U transits to the shallow sleep state), when the first temperature is the bed ambient temperature before user U gets into bed, starts sleeping, or falls asleep (in the present embodiment, before user U starts sleeping).

Here, the shallow sleep state indicates a state of depth of sleep of user U in shallow sleep such that a voice or a sound readily wakes user U. The expression “before the non-shallow sleep state ends” indicates a period in the sleep cycle after the user falls asleep and transits to the first non-shallow sleep state and before the user transits to the shallow sleep state.

For example, sensor 200 may further include a sleep sensor for detecting whether user U is in shallow sleep or in non-shallow sleep. For example, the sleep sensor is any sensor which detects the sleep state of user U by detecting tossing and turning, breathing, the heart rate of user U, such as a vibration detecting sensor or a radiowave sensor.

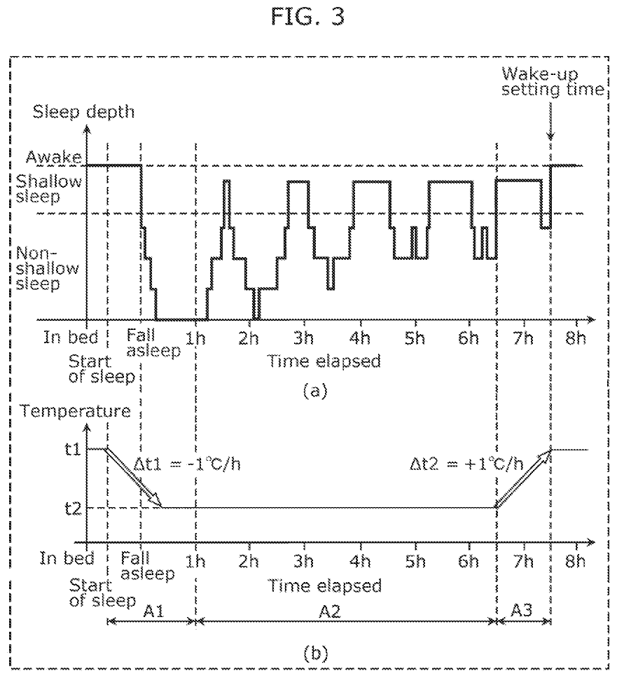

FIG. 3 is a graph schematically illustrating one example of the sleep cycle (sleep curve). Specifically, (a) of FIG. 3 is a graph schematically illustrating one example of the sleep cycle of user U, and (b) of FIG. 3 is a graph schematically illustrating one example of a change in bed ambient temperature. In the graph illustrated in (a) of FIG. 3, the abscissa represents the time elapsed since user U has fallen asleep, and the ordinate represents the sleep depth (sleep stage) of user U. The sleep depth is a degree of the depth of sleep. A upper level of sleep depth (upper side of the graph) indicates shallower sleep of user U, and a lower level of sleep depth (lower side of the graph) indicates deeper sleep of user U.

In the sleep cycle, alternation of the shallow sleep and the non-shallow sleep occurs approximately every 90 minutes. User U gets into a non-shallow sleep state once after the user falls asleep, and further gets into a shallow sleep state after approximately 90 minutes after the user falls asleep. In the shallow sleep state, a change in external environment more readily wakes user U than in the non-shallow sleep state. To prevent user U from waking, by controlling temperature adjusting apparatus 300, control apparatus 100 decreases the bed ambient temperature by 0.5° C. or more in a period after user U falls asleep and before user U gets into the shallow sleep state. For this reason, the period before user U transits to the shallow sleep state may be within 90 minutes since user U has fallen asleep, for example. Alternatively, the period may be within 60 minutes after user U has fallen asleep. These times are only one examples, and may have errors of about 10%.

For example, as illustrated in zone A1 in (b) of FIG. 3, by controlling temperature adjusting apparatus 300, controller 130 changes the bed ambient temperature from the first temperature (temperature t1) to the second temperature (temperature t2) such that the change in temperature is 1° C./h (temperature change amount Δt1). Subsequently, for example, as illustrated in zone A2 in (b) of FIG. 3, controller 130 controls temperature adjusting apparatus 300 such that the second temperature does not change.

The first temperature is a temperature measured by sensor 200, for example. Alternatively, in the case where user U preliminarily operates temperature adjusting apparatus 300, the first temperature may be a temperature which control apparatus 100 estimates based on the information indicating the current operation aspect, which is obtained from temperature adjusting apparatus 300, and setting information 141.

The second temperature is a temperature calculated from the first temperature by control apparatus 100, for example. The second temperature is lower than the first temperature by at least 0.5° C. and at most 1.5° C. (more specifically, 1° C.). In the case where temperature adjusting apparatus 300 is preliminarily operated before user U starts sleeping and the first temperature is predetermined, the second temperature information indicating the second temperature may be preliminarily stored in storage 140.

For example, in the case where controller 130 changes the bed ambient temperature from the first temperature to the second temperature by controlling temperature adjusting apparatus 300, controller 130 changes the bed ambient temperature in the range of −1.5° C./h or more and −0.5° C./h or less.

For example, in the case where controller 130 changes the bed ambient temperature from the first temperature to the second temperature by controlling temperature adjusting apparatus 300, controller 130 controls the bed ambient temperature such that the change amount thereof is fixed against time.

For example, in the case where obtainer 110 obtains the third temperature information including the third temperature which is the temperature when user U is sleeping, controller 130 changes the bed ambient temperature from the second temperature to the third temperature within 60 minutes after changing the bed ambient temperature to the second temperature. For example, controller 130 changes the bed ambient temperature from the second temperature to the third temperature and then maintains the bed ambient temperature at the third temperature.

For example, when obtainer 110 obtains the wake-up setting information including the wake-up setting time of user U, controller 130 changes the bed ambient temperature over 60 minutes up to the wake-up setting time such that the bed ambient temperature increases by at least 0.5° C. and at most 1.5° C.

For example, as illustrated in zone A3 in (b) of FIG. 3, by controlling temperature adjusting apparatus 300, controller 130 increases the bed ambient temperature from the second temperature over 60 minutes such that a change in temperature is 1° C./h (temperature change amount Δt2).

Alternatively, in the case where obtainer 110 obtains the third temperature information and the wake-up setting information, controller 130 changes the bed ambient temperature over 60 minutes up to the wake-up setting time to cause the bed ambient temperature at the wake-up setting time to reach a fourth temperature which is higher than the third temperature by at least 0.5° C. and at most 1.5° C.

The fourth temperature is a temperature calculated by controller 130 based on the third temperature.

For example, controller 130 is implemented by a communication interface for communicating with temperature adjusting apparatus 300, the control program stored in storage 140, and the CPU which executes the control program.

For example, storage 140 is a recording apparatus which stores the control programs executed by estimator 120 and controller 130, setting information 141, and the like. Storage 140 is a flash memory or a hard disk drive (HDD), for example.

### [Operation]

The operation of control apparatus 100 having such a configuration will now be described.

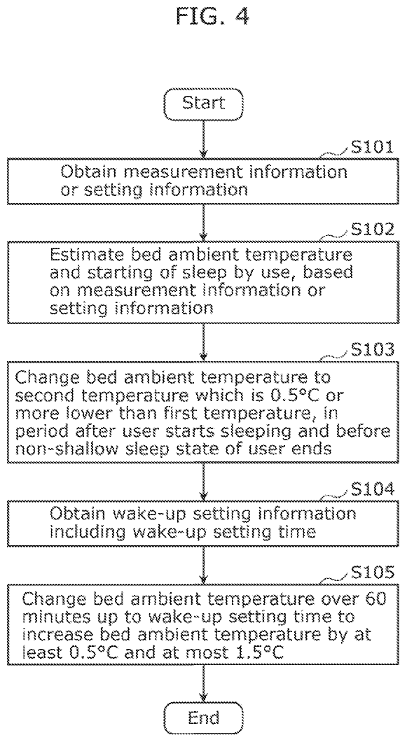

FIG. 4 is a flowchart illustrating the processing procedure in control apparatus 100 according to the embodiment.

Initially, obtainer 110 obtains the measurement information from sensor 200 or setting information 141 of temperature adjusting apparatus 300 from storage 140 (step S101).

Next, estimator 120 estimates the bed ambient temperature and the starting of sleep by user U based the measurement information or setting information 141 obtained by obtainer 110 (step S102).

Next, when the first temperature is the bed ambient temperature before user U starts sleeping, controller 130 compares the bed ambient temperature to the first temperature and changes the bed ambient temperature to the second temperature, which is 0.5° C. or more lower than the bed ambient temperature, in the period after user U starts sleeping and before user U gets into the non-shallow sleep state (step S103).

For example, when the third temperature is higher than or equal to the second temperature and lower than or equal to the first temperature, controller 130 changes the bed ambient temperature to the second temperature, which is lower than the first temperature by at least 0.5° C. and at most 1.5° C., in the period after user U starts sleeping and before user U gets into the non-shallow sleep state. For example, when the third temperature is lower than or equal to the second temperature, controller 130 changes the bed ambient temperature to the second temperature, which is lower than the first temperature by 1.5° C. or more (for example, the same temperature as the third temperature), in the period after user U starts sleeping and before user U gets into the non-shallow sleep state.

Details of the processing performed on the third temperature by controller 130 will be described later.

For example, controller 130 changes the bed ambient temperature from the first temperature to the second temperature within 90 minutes after user U starts sleeping. For example, controller 130 changes the bed ambient temperature from the first temperature to the second temperature within 60 minutes after user U starts sleeping.

As the bed ambient temperature changes more mildly, user U can have more comfortable sleep. For this reason, controller 130 controls temperature adjusting apparatus 300 such that the change amount in bed ambient temperature is fixed against the time (for example, temperature change amount Δt1 indicating the slope of the graph illustrated in FIG. 3 is fixed), for example.

Next, obtainer 110 obtains the wake-up setting information including the wake-up setting time of user U (step S104).

Next, by controlling temperature adjusting apparatus 300, controller 130 changes the bed ambient temperature over 60 minutes up to the wake-up setting time included in the wake-up setting information obtained by obtainer 110 such that the bed ambient temperature is decreased by at least 0.5° C. and at most 1.5° C. (step S105). For example, controller 130 controls temperature adjusting apparatus 300 such that the change amount in bed ambient temperature is fixed against the time (for example, temperature change amount Δt1 illustrated in FIG. 3 is fixed).

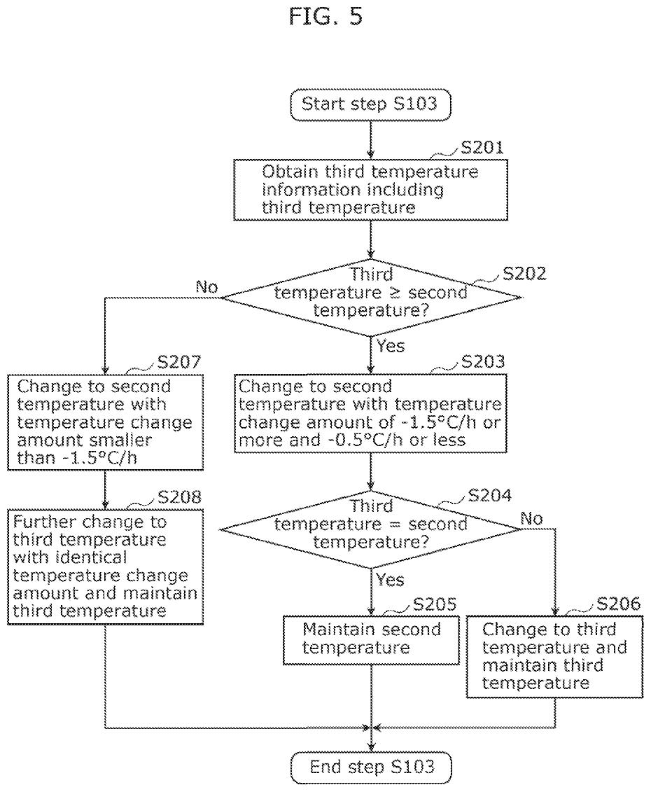

FIG. 5 is a flowchart illustrating details of the temperature control processing procedure (step S103 illustrated in FIG. 4) in control apparatus 100 according to the embodiment.

As described above, controller 130 controls the bed ambient temperature, for example, based on the third temperature information including the third temperature which is the temperature during sleeping of user U obtained by obtainer 110.

Initially, obtainer 110 obtains the third temperature information including the third temperature which is the temperature during sleeping of user U (step S201).

Next, estimator 120 determines whether the third temperature included in the third temperature information obtained by obtainer 110 is higher than or equal to the second temperature (step S202). For example, estimator 120 estimates the first temperature based on the measurement information or the setting information obtained by obtainer 110 in step S101, and then estimates the second temperature from the estimated first temperature. The second temperature is calculated by an expression (first temperature −1° C.), for example.

When estimator 120 determines that the third temperature is higher than or equal to the second temperature (Yes in step S202), by controlling temperature adjusting apparatus 300, controller 130 changes the bed ambient temperature from the first temperature to the second temperature with a temperature change amount of −1.5° C./h or more and −0.5° C./h or less (step S203).

Next, estimator 120 determines whether the third temperature included in the third temperature information obtained by obtainer 110 is equal to the second temperature (step S204).

When estimator 120 determines that the third temperature is equal to the second temperature (Yes in step S204), by controlling temperature adjusting apparatus 300, controller 130 maintains the bed ambient temperature at the second temperature (=third temperature) (step S205).

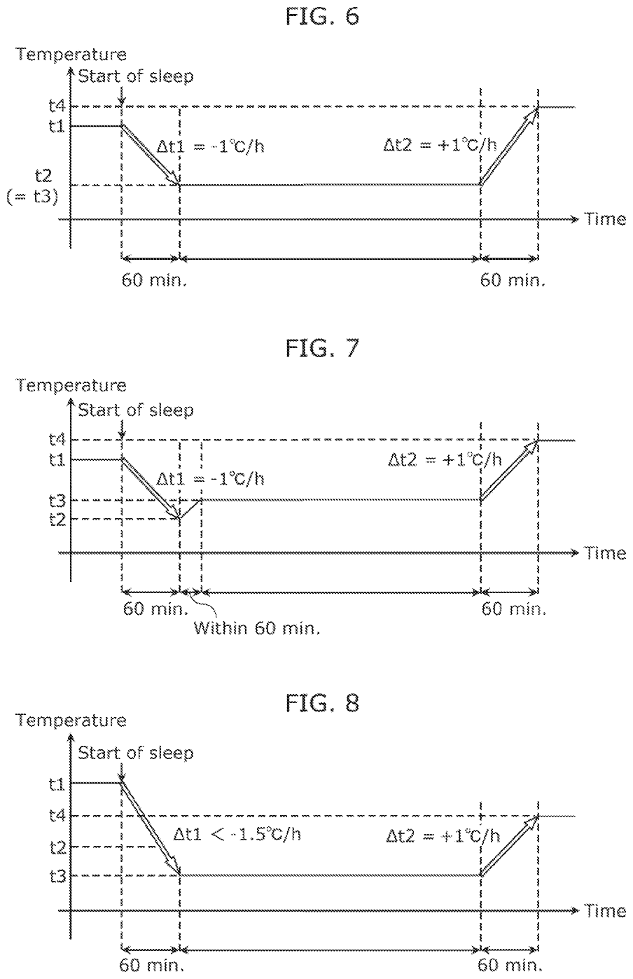

FIG. 6 is a graph illustrating a first example of a time change in bed ambient temperature controlled by control apparatus 100 according to the embodiment. Specifically, FIG. 6 is a graph illustrating a time change in bed ambient temperature when control apparatus 100 executes steps S201, S202, S203, S204, and S205.

As illustrated in FIG. 6, for example, by controlling temperature adjusting apparatus 300, controller 130 changes the bed ambient temperature from the first temperature (temperature t1) to the second temperature (temperature t2) at the timing when user U starts sleeping, and maintains the bed ambient temperature at the second temperature.

Again with reference to FIG. 5, when estimator 120 determines that the third temperature is not equal to the second temperature (No in step S204), by controlling temperature adjusting apparatus 300, controller 130 changes the bed ambient temperature from the second temperature to the third temperature, and maintains the bed ambient temperature at the third temperature (step S206). The temperature change amount in changing from the second temperature to the third temperature is not particularly limited. For example, by controlling temperature adjusting apparatus 300, controller 130 changes the bed ambient temperature to the third temperature within 60 minutes from the changing of the bed ambient temperature to the second temperature.

FIG. 7 is a graph illustrating a second example of a time change in bed ambient temperature controlled by control apparatus 100 according to the embodiment. Specifically, FIG. 7 is a graph illustrating a time change in bed ambient temperature when control apparatus 100 executes steps S201, S202, S203, S204, and S206.

As illustrated in FIG. 7, for example, by controlling temperature adjusting apparatus 300, controller 130 changes the bed ambient temperature from the first temperature (temperature t1) to the second temperature (temperature t2) at the timing when user U starts sleeping, further changes it from the second temperature to the third temperature (temperature t3), and then maintains the bed ambient temperature at the third temperature.

Again with reference to FIG. 5, when estimator 120 determines that the third temperature is less than the second temperature (No in step S202), by controlling temperature adjusting apparatus 300, controller 130 changes the bed ambient temperature from the first temperature to the second temperature with a temperature change amount smaller than −1.5° C./h (step S207).

Next, by controlling temperature adjusting apparatus 300, controller 130 changes the bed ambient temperature from the second temperature to the third temperature with the same temperature change amount as that in step S207, and maintains the bed ambient temperature at the third temperature (step S208).

FIG. 8 is a graph illustrating a third example of a time change in bed ambient temperature controlled by control apparatus 100 according to the embodiment. Specifically, FIG. 8 is a graph illustrating a time change in bed ambient temperature when control apparatus 100 executes steps S201, S202, S207, and S208.

As illustrated in FIG. 8, for example, by controlling temperature adjusting apparatus 300, controller 130 changes the bed ambient temperature from the first temperature (temperature t1) to the second temperature (temperature t2) at the timing when user U starts sleeping, further changes it from the second temperature to the third temperature (temperature t3), and then maintains the bed ambient temperature at the third temperature. Here, in this example, by controlling temperature adjusting apparatus 300, for example, controller 130 changes the bed ambient temperature from the first temperature to the third temperature at the timing when user U starts sleeping such that the change amount in bed ambient temperature is fixed against the time (for example, temperature change amount Δt2 illustrated in FIG. 8 is fixed).

Thus, controller 130 appropriately controls the bed ambient temperature by controlling temperature adjusting apparatus 300 based on the third temperature.

For example, in step S105, by controlling temperature adjusting apparatus 300, controller 130 may change the bed ambient temperature over 60 minutes up to the wake-up setting time to cause the bed ambient temperature at the wake-up setting time to reach a fourth temperature (temperature t4 illustrated in FIGS. 6 to 8) which is higher than the third temperature by at least 0.5° C. and at most 1.5° C. For example, controller 130 controls temperature adjusting apparatus 300 such that the change amount in bed ambient temperature is fixed against the time (for example, temperature change amount Δt2 illustrated in FIGS. 6 to 8 is fixed).

### [Experimental Results]

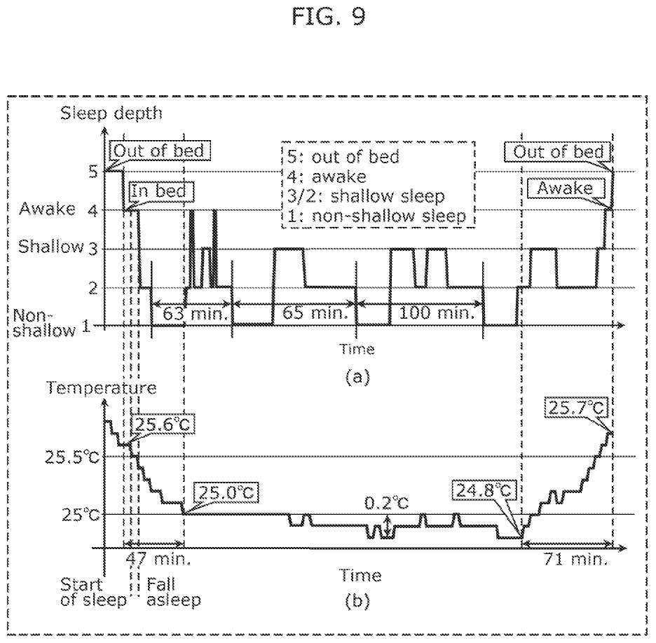

FIG. 9 is a graph illustrating an experimental result of the time change in sleep depth of user U and the time change in bed ambient temperature controlled by control apparatus 100 according to the embodiment. Specifically, (a) of FIG. 9 is a graph illustrating an experimental result of the time change in sleep depth of user U, and (b) of FIG. 9 is a graph illustrating the time change in bed ambient temperature controlled by control apparatus 100 according to the embodiment when the experimental result illustrated in (a) of FIG. 9 is obtained.

In the graph illustrated in (a) of FIG. 9, the ordinate represents the sleep depth of user U. Stage 1 indicates that user U is in non-shallow sleep. Stages 2 and 3 indicate that user U is in shallow sleep. Stage 4 indicates that user U gets into bed and is awake or wakes. Stage 5 indicates that user U is out of bed.

In the example illustrated in FIG. 9, control apparatus 100 executes steps S201, S202, S203, S204, and S205 described above.

As illustrated in FIG. 9, by controlling temperature adjusting apparatus 300, control apparatus 100 changes the bed ambient temperature from 25.6° C. to 25.0° C. over 47 minutes after user U gets into bed. Thereby, user U appropriately transits from the awake state to the non-shallow sleep state.

By controlling temperature adjusting apparatus 300, control apparatus 100 maintains the bed ambient temperature at 25.0° C.±0.2° C. Thereby, alternation of shallow sleep and non-shallow sleep appropriately occurs in user U.

Thus, control apparatus 100 can bring user U into a comfortable sleep state.

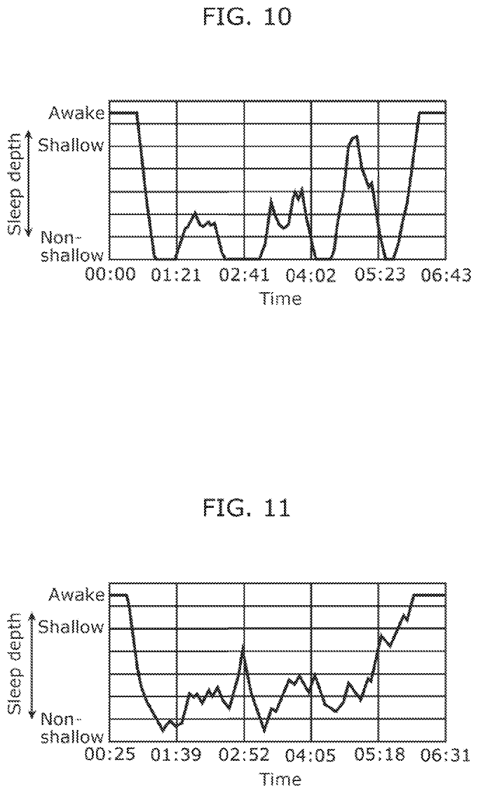

FIG. 10 is a graph illustrating another example of the experimental result of the time change in sleep depth of user U in control apparatus 100 according to the embodiment. FIG. 11 is a graph illustrating another example of the experimental result of the time change in sleep depth of user U according to Comparative Example. The graph according to Comparative Example illustrated in FIG. 11 illustrates the experimental result of the time change in sleep depth of user U when the bed ambient temperature is not particularly controlled.

FIG. 10 shows that alternation of a low state and a high state clearly occurs in the sleep depth of user U when the bed ambient temperature is controlled by control apparatus 100. This indicates that user U is in a comfortable sleep state because alternation of the shallow sleep and the non-shallow sleep appropriately occurs.

In Comparative Example illustrated in FIG. 11, the experimental result shows that the sleep depth of user U is not as deep as that in the experimental result illustrated in FIG. 10, and clear alternation of a low state and a high state does not occur. This indicates that user U is not in a comfortable sleep state because alternation of the shallow sleep and the non-shallow sleep does not appropriately occur.

### [Effects]

As described above, the control method according to one aspect of the present disclosure is a control method for an apparatus (such as temperature adjusting apparatus 300) which adjusts the ambient temperature of the bed of user U, the control method including: obtaining one of measurement information in the sensor (such as sensor 200) and the setting information of apparatus 141 (step S101); estimating the bed ambient temperature and one of getting in bed, starting of sleep, and falling asleep by user U, based on the one of the measurement information and setting information 141 (step S102); and when the first temperature is the bed ambient temperature before user U gets into bed, starts sleeping, or falls asleep, changing the bed ambient temperature to a second temperature, which is lower than the first temperature by 0.5° C. or more, in a period after user U gets into bed, starts sleeping, or falls asleep and before a non-shallow sleep state of user U ends (step S103). For example, the control method according to one aspect of the present disclosure estimates the bed ambient temperature and getting into bed by user U, based on the one of the measurement information and setting information 141, and changes the bed ambient temperature to the second temperature, which is lower than the first temperature by 0.5° C. or more, in a period after user U gets into bed and before the non-shallow sleep state of user U ends, when the first temperature is the bed ambient temperature before user U gets into bed. Alternatively, the control method according to one aspect of the present disclosure estimates the bed ambient temperature and starting of sleep by user U, based on the one of the measurement information and setting information 141, and changes the bed ambient temperature to the second temperature, which is lower than the first temperature by 0.5° C. or more, in a period after user U starts sleeping and before the non-shallow sleep state of user U ends, when the first temperature is the bed ambient temperature before user U starts sleeping. Alternatively, the control method according to one aspect of the present disclosure estimates the bed ambient temperature and falling asleep by user U, based on the one of the measurement information and setting information 141, and changes the bed ambient temperature to the second temperature, which is lower than the first temperature by 0.5° C. or more, in a period after user U falls asleep and before the non-shallow sleep state of user U ends, when the first temperature is the bed ambient temperature before user U falls asleep.

Thereby, the bed ambient temperature can be decreased by 0.5° C. or more at the timing when user U gets into bed, starts sleeping, or falls asleep. For example, it is considered that a decrease in bed ambient temperature by 1° C. after user U closes eyes can trigger autonomous decrease of the deep body temperature of user U. For this reason, this control method can cause user U to appropriately fall asleep. In the related art, there is a method of continuously decreasing the temperature of the room where user U sleeps, in order to promote falling asleep by user U. Continuous decrease of the room temperature causes significant cold, which obstructs maintenance of the sleep state of user U. In particular, in the shallow sleep state, user U is more likely to wake up due to a change in external environment than when user U is in the non-shallow sleep state. For this reason, while the method in the related art can appropriately cause user U to fall asleep, the method cannot ensure comfortable sleep thereafter. Moreover, the method in the related art only continuously decreases the temperature of the room where user U sleeps, and does not always appropriately decrease the bed ambient temperature, which can appropriately promote falling asleep by user U. Thus, the control method according to the present disclosure decreases the bed ambient temperature by 0.5° C. or more in a period after user U gets into bed, starts sleeping, or falls asleep and before the non-shallow sleep state of user U ends. This can appropriately promote falling asleep by user U, and can prevent user U from readily waking up after the user falls asleep. In other words, the control method according to one aspect of the present disclosure can promote a more comfortable sleep state of user U than in the related art. The control method according to the present disclosure can also promote a more comfortable sleep state of user U irrespective of the seasons.

Moreover, for example, the control method according to the present disclosure further obtains the third temperature information including the third temperature which is the temperature when user U is sleeping (step S201), and changes the bed ambient temperature from the second temperature to the third temperature within 60 minutes after changing the bed ambient temperature to the second temperature (e.g., step S206).

This can appropriately cause user U to fall asleep and to sleep at a temperature which user U likes.

Moreover, for example, the control method according to the present disclosure further obtains the wake-up setting information including the wake-up setting time of user U (step S104), and changes the bed ambient temperature over 60 minutes up to the wake-up setting time to cause the bed ambient temperature at the wake-up setting time to reach the fourth temperature which is higher than the third temperature by at least 0.5° C. and at most 1.5° C.

Alternatively, for example, the control method according to the present disclosure changes the bed ambient temperature over 60 minutes up to the wake-up setting time to increase the bed ambient temperature by at least 0.5° C. and at most 1.5° C. (step S105).

It is considered that an autonomous increase of the deep body temperature in user U can be triggered by gradually increasing the bed ambient temperature by about 1° C. before about 60 minutes when user U gets up. Here, a sharp increase in bed ambient temperature may awaken user U in sudden due to significant heat, impairing a comfortable feel in wake-up. Thus, user U can wake up with a comfortable feel by gradually increasing the bed ambient temperature by about 1° C. before 60 minutes when user U gets up.

Moreover, for example, the control method according to the present disclosure further changes the bed ambient temperature from the second temperature to the third temperature, and then maintains the bed ambient temperature at the third temperature (step S205, S206, or S208).

This method can hardly cause user U to feel a change in temperature to awaken user U, and thus can readily maintain a comfortable sleep state of user U.

Moreover, for example, the changing of the bed ambient temperature to the second temperature (step S103) includes controlling the change amount of the bed ambient temperature against time to be fixed.

When the bed ambient temperature is sharply changed, user U may feel the change in temperature obstructive and cannot appropriately fall asleep. For this reason, this method can cause user U to appropriately fall asleep while hardly causing user U to feel such a change in temperature.

Moreover, for example, the changing of the bed ambient temperature to the second temperature (step S103) includes changing the bed ambient temperature with an change amount of −1.5° C./h or more and −0.5° C./h or less.

This can cause user U to fall asleep without feeling cold and feeling hot.

Moreover, for example, in the above embodiment, all or part of components such as control apparatus 100 may be configured with dedicated hardware, or may be implemented by executing software programs suitable for the components. The components may be implemented by a program executor such as a CPU or a processor, which reads out and executes software programs recorded in a recording medium such as a HDD or a semiconductor memory. Here, the software which implements the information processing system according to the embodiment above is a program as follows.

In other words, this program is a program causing a computer to execute the control method described above, the program including: obtaining one of the measurement information in the sensor (e.g., sensor 200) and setting information 141 of the apparatus (step S101); estimating the bed ambient temperature and one of getting in bed, starting of sleep, and falling asleep by user U, based on the one of the measurement information and setting information 141 (step S102); and when the first temperature is the bed ambient temperature before user U gets into bed, starts sleeping, or falls asleep, changing the bed ambient temperature to a second temperature, which is lower than the first temperature by 0.5° C. or more, in a period after user U gets into bed, starts sleeping, or falls asleep and before a non-shallow sleep state of user U ends (step S103).

This ensures the same effects as those in the control method according to the present disclosure.

Moreover, control apparatus 100 according to one aspect of the present disclosure is a control apparatus for the apparatus (e.g., temperature adjusting apparatus 300) which adjusts the ambient temperature of the bed of user U, and includes: obtainer 110 which obtains one of the measurement information in the sensor (such as sensor 200) and setting information 141 of the apparatus; estimator 120 which estimates the bed ambient temperature and one of getting in bed, starting of sleep, and falling asleep by user U, based on the one of the measurement information and setting information 141; and controller 130 which changes the bed ambient temperature to the second temperature, which is lower than the first temperature by 0.5° C. or more, in a period after user U gets into bed, starts sleeping, or falls asleep and before the non-shallow sleep state of user U ends, when the first temperature is the bed ambient temperature before user U gets into bed, starts sleeping, or falls asleep.

This ensures the same effects as those in the control method according to the present disclosure.

### [Other Embodiments]

Although the control method and the like according to one or a plurality of aspects of the present disclosure have been described as above based on the embodiment, this embodiment should not be construed as limitations to the present disclosure. One or a plurality of aspects of the present disclosure may also cover a variety of modifications of the present embodiment conceived and made by persons skilled in the art without departing from the gist of the present disclosure, and combinations of components in different embodiments.

For example, obtainer 110 may obtain the wake-up setting information in step S101, rather than in step S104.

Moreover, for example, obtainer 110 may obtain the third temperature information in step S101, rather than in step S201.

Moreover, for example, when the bed ambient temperature is maintained at the second temperature or the third temperature, controller 130 had not better to cause temperature adjusting apparatus 300 to change the air amount or the wind direction, to stop and start the operation, and to switch between cooling and heating. Thereby, controller 130 can suppress generation of sounds by temperature adjusting apparatus 300, and therefore, can prevent user U from waking up in the state where the bed ambient temperature is maintained at the second temperature or the third temperature.

As described above, the embodiments above are all one specific examples of the present disclosure. Accordingly, numeric values, shapes, materials, components, arrangements of components, connection forms thereof, steps, order of the steps, and the like shown in the embodiments above are exemplary, and should not be construed as limitations to the present disclosure.

Moreover, for example, in the embodiments above, all or part of the components in the control apparatus according to the present disclosure may be configured with dedicated hardware, or may be implemented by executing software programs suitable for the components. The components may be implemented by a program executor such as a CPU or a processor which reads out and executes software programs recorded in a recording medium such as a HDD or a semiconductor memory.

Moreover, the components in the control apparatus according to the present disclosure may be configured with one or a plurality of electronic circuits. One or a plurality of electronic circuits may be general-purpose circuits, or may be dedicated circuits.

One or a plurality of electronic circuits may include a semiconductor device, an integrated circuit (IC), or large scale integration (LSI), for example. IC or LSI may be integrated into a single chip, or may be integrated into a plurality of chips. Here, although the term IC or LSI is used, these electronic circuits are called in different names depending on the degree of integration, and may be called system LSI, very large scale integration (VLSI), or ultra large scale integration (ULSI) in some cases. A field programmable gate array (FPGA) programed after manufacturing of LSI can also be used for the same purpose.

Moreover, general or specific aspects of the present disclosure may be implemented with a system, a device, a method, an integrated circuit, or a computer program. Alternatively, general or specific aspects of the present disclosure may be implemented with a computer-readable non-transitory recording medium having the computer program recorded thereon, such as an optical disc, a HDD, or a semiconductor memory. Such general or specific aspects of the present disclosure may be implemented with any combination of a system, a device, a method, an integrated circuit, a computer program, and a recording medium.

Besides, the present disclosure also covers embodiments obtained by modifying the embodiments in various ways conceived by persons skilled in the art, and embodiments implemented by any combination of the components and the functions included in the above embodiments without departing from the gist of the present disclosure.

### INDUSTRIAL APPLICABILITY

The present disclosure is applicable to control apparatuses which control apparatuses which can control bed ambient temperatures of users. Specifically, the present disclosure is applicable to air-conditioning apparatuses and the like.

1. A control method for an apparatus which adjusts a bed ambient temperature for a user, the control method comprising:
obtaining one of measurement information in a sensor and setting information of the apparatus;
estimating the bed ambient temperature and one of getting in bed, starting of sleep, and falling asleep by the user, based on the one of the measurement information and the setting information; and
when a first temperature is the bed ambient temperature before the user gets into bed, starts sleeping, or falls asleep, changing the bed ambient temperature to a second temperature, which is lower than the first temperature by 0.5° C. or more, in a period after the user gets into bed, starts sleeping, or falls asleep and before a non-shallow sleep state of the user ends.

2. The control method according to claim 1, further comprising:
obtaining third temperature information including a third temperature which is a temperature when the user is sleeping; and
changing the bed ambient temperature from the second temperature to the third temperature within 60 minutes after changing the bed ambient temperature to the second temperature.

3. The control method according to claim 2, further comprising:
obtaining wake-up setting information including wake-up setting time of the user; and
changing the bed ambient temperature over 60 minutes up to the wake-up setting time to cause the bed ambient temperature at the wake-up setting time to reach a fourth temperature which is higher than the third temperature by at least 0.5° C. and at most 1.5° C.

4. The control method according to claim 2, further comprising:
changing the bed ambient temperature from the second temperature to the third temperature, and then maintaining the bed ambient temperature at the third temperature.

5. The control method according to claim 1, further comprising:
obtaining wake-up setting information including wake-up setting time of the user; and
changing the bed ambient temperature over 60 minutes up to the wake-up setting time to increase the bed ambient temperature by at least 0.5° C. and at most 1.5° C.

6. The control method according to claim 1,
wherein the changing of the bed ambient temperature to the second temperature includes controlling a change amount of the bed ambient temperature against time to be fixed.

7. The control method according to claim 1,
wherein the changing of the bed ambient temperature to the second temperature includes changing the bed ambient temperature with a change amount of −1.5° C./h or more and −0.5° C./h or less.

8. A non-transitory computer-readable recording medium for use in a computer, the recording medium having a computer program recorded thereon for causing the computer to execute the control method according to claim 1.

9. A control apparatus for an apparatus which adjusts a bed ambient temperature for a user, the control apparatus comprising:
an obtainer which obtains one of measurement information in a sensor and setting information of the apparatus;
an estimator which estimates the bed ambient temperature and one of getting in bed, starting of sleep, and falling asleep by the user, based on the one of the measurement information and the setting information; and
a controller which causes the apparatus to change the bed ambient temperature to a second temperature, which is lower than a first temperature by 0.5° C. or more, in a period after the user gets into bed, starts sleeping, or falls asleep and before a non-shallow sleep state of the user ends, when the first temperature is the bed ambient temperature before the user gets into bed, starts sleeping, or falls asleep.
